SEQUENCE LISTING (6 sequences)

SEQ ID NO: 1 - DNA, 31 bp, Artificial
  primer
tctgaattcgcagcagcgccggtataagcac

SEQ ID NO: 2 - DNA, 28 bp, Artificial
  primer
tacgtaatcgccagcgcagcggccagtt

SEQ ID NO: 3 - DNA, 248 bp, Artificial
  promoter
cagcagcgccggtataagcaccggcgccacattgcgttatcagcgagcgctggtcgatctggaggtcagccgcggctggattttgtctaaccacgctacgccggaagatcccgttcaggtgttggcccgcttttcttacaccttttaatcaacagttttgcaggaccccacggcaatatacggagagacatggatgaaaaataataacttcagactttcggcggcaggcaaactggccgctgcgctgg

SEQ ID NO: 4 - DNA, 30 bp, Artificial
  primer
ccggaattcgccggttaccaatgccacttt

SEQ ID NO: 5 - DNA, 28 bp, Artificial
  primer
taatacgtagggggagcgaaacatgcag

SEQ ID NO: 6 - DNA, 300 bp, Artificial
  promoter
acgctgctcgttttgttgctgttgtcttttcgcggcgatgcgcagagccatagccacggcggccatcatgcttccgctttgcagcgggtacatcaggcggcggcgacggatgcttacggctgcgccaagtcgcgcattaaaaagcagctgcgccgcctggctcagctttgaagcggtttttagggagccgctctatactctgacgggcatacggggcgcctgcgcgcaggccgctgtgttgatatcgctaaccagagaaaaaacagtatgacgaaagtggcattggtaaccggcgcaagc

(19) United States
(12) Patent Application Publication
(10) Pub. No.: US 2005/0136445 A1
(43) Pub. Date: Jun. 23, 2005

(54) Recombinant DNA vector, recombinant bio-luminescent bacterium and method for detecting bio-inhibitory substances using same

(75) Inventors: Show-Ying Yang, Taipei City (TW); Hsin-Chih Lai, Taipei County (TW); Jun-Rong Wei, Taipei City (TW); Hui-Ling Lin, Hsinchu City (TW)

Correspondence Address: BACON & THOMAS, PLLC, 625 SLATERS LANE, FOURTH FLOOR, ALEXANDRIA, VA, 22314, US

(73) Assignee: Industrial Technology Research Institute, Hsin Chu (TW)

(21) Appl. No.: 10/950,417

(22) Filed: Sep. 28, 2004

(30) Foreign Application Priority Data
Dec. 19, 2003 (TW) 92136311

(51) Int. Cl. C12Q 1/68; C12N 15/74; C12N 1/21

(52) U.S. Cl. 435/6; 435/471; 435/252.3

(57) ABSTRACT

The present invention relates to recombinant DNA vector, recombinant bio-luminescent bacterium and method for detecting bio-inhibitory substances by using foregoing bacterium. Since bio-inhibitory substances can inhibit or stimulate the luminescence of recombinant bio-luminescent bacterium, the invention can be applied in detection of the presence and amount of toxic substances in waste water.

### BACKGROUND OF THE INVENTION

1. Field of the Invention

The present invention relates to recombinant DNA vector, recombinant bio-luminescent bacterium and method for detecting bio-inhibitory substances using the foregoing bacterium.

2. Description of Related Art

When the process in a factory goes wrong, it often results in the discharge of large amount of highly concentrated waste fluid or wastewater. Many factories have regulating pond installed to act as a buffer in such incidences. But constrained by limited land, such regulating pond usually does not work as well as expected. As a result, the wastewater treatment system is heavily burdened or has too many inhibitory and toxic substances flowed in. For water quality monitoring, replacing traditional external environmental indicators with biochemical indicators could help understand changes of wastewater characteristics. In particular if the wastewater contains substances toxic to organisms, the use of biochemical indicators can reflect instantly the content and impact of such substances to help manage the treatment and discharge of wastewater and maintain the efficacy and stability of the overall treatment system. Usually if a treatment system is overwhelmed with biological toxicants, it will take a long time to restore its original functions or take the addition of biological agents to temporarily sustain its functions. Thus finding appropriate biochemical indicators poses a pressing need for the biological wastewater treatment system.

In the process of biological wastewater treatment, the most ideal monitoring system is a system that is capable of monitoring all kinds of components in the wastewater, including the monitoring of possible toxic substances and their concentration, and allows long-term analysis and the establishment of database to achieve the objectives of monitoring and maintaining system stability. Sampling wastewater periodically and subjecting it to laboratory pretreatment and then using gas or liquid chromatography and mass spectrography for analysis is a feasible approach. But chromatography is not necessarily applicable at all wastewater treatment plants, in particular if the components of wastewater are complex and vary wildly, which makes the pretreatment process and the operation and maintenance of the system tedious and complicated. The required facilities would be expensive and are not necessarily appropriate for toxicity testing. Moreover, the determination of wastewater components does not directly reveal the biotoxicity of those components, hence bringing about the assay of bio-inhibitory substance. Observing the reaction of a species towards certain substance (or wastewater), i.e. toxicity monitoring may be used to assess water quality. Toxicity assessment used to be applied in pharmaceutical industry and public hazard prevention only. Now it has been extended to the monitoring of wastewater toxicity, in which organisms such as fathead minnow, water flea, daphnia and mysid shrimp are commonly used.

The techniques detecting bio-inhibitory substances in wastewater have much room for improvement. The main goal is to detect instantly the type, concentration and impact of inhibitory or toxic substance in the wastewater. So far many methods have been developed, for example, using naturally luminescent bacterium Vibrio fischeri and the principle that the light emission intensity of the bacterium is inversely proportional to the concentration of toxicant to monitor toxic substances in wastewater. A drawback of such monitoring system is that Vibrio fischeri must be cultured near marine environment, rendering the culturing of bacterium inconvenient and hence limiting its applications. Thus there is a pressing need to build a fast and convenient system for detecting bio-inhibitory substances in wastewater.

### SUMMARY OF THE INVENTION

To address the drawbacks of known techniques for detecting bio-inhibitory substances, the present invention provides recombinant DNA vector, recombinant bio-luminescent bacterium and method for detecting bio-inhibitory substances using the foregoing bacterium to achieve better efficiency and convenience for monitoring the quality of wastewater.

In one aspect, the present invention relates to a recombinant DNA vector, comprising an environmental sensitive promoter and a reporter. In one embodiment, the foregoing recombinant DNA vector consists of an environmental sensitive promoter of the full-length sh1A gene, a fragment, derivative, or degenerate sequence thereof; and a reporter, comprising a light-emitting protein encoding gene positioned in-frame downstream of the foregoing promoter.

In another embodiment, the foregoing recombinant DNA vector consists of an environmental sensitive promoter of the full-length fabG gene, a fragment, derivative, or degenerate sequence thereof; and a reporter, comprising a light-emitting protein encoding gene positioned in-frame downstream of the foregoing promoter.

Another objective of the present invention is to provide a recombinant bio-luminescent bacterium which carries aforesaid recombinant DNA vector having environmental sensitive promoter.

In one embodiment, the foregoing recombinant bio-luminescent bacterium carries a recombinant DNA vector having environmental sensitive promoter which contains the full-length sh1A gene promoter, a fragment, derivative, or degenerate sequence thereof; and a reporter, comprising a light-emitting protein encoding gene positioned in-frame downstream of abovementioned promoter; the foregoing recombinant bio-luminescent bacterium has been preserved at the Culture Collection and Research Center of the Food Industry Research and Development Institute (No. BCRC940434) since Oct. 17, 2003.

In another embodiment, the foregoing recombinant bio-luminescent bacterium carries a recombinant DNA vector having environmental sensitive promoter of the full-length fabG gene, a fragment, derivative, or degenerate sequence thereof; and a reporter, comprising a light-emitting protein encoding gene positioned in-frame downstream of said promoter; the foregoing recombinant bio-luminescent bacterium has been preserved at the Culture Collection and Research Center of the Food Industry Research and Development Institute (No. BCRC940433) since Oct. 17, 2003.

The present invention also relates to a recombinant bio-luminescent bacterium for detecting bio-inhibitory substances carrying the foregoing recombinant DNA vector, wherein the aforesaid recombinant bio-luminescent bacterium can sense the concentration of bio-inhibitory substances such that the higher the concentration of such substances, the lower its luminosity.

A further objective of the present invention is to provide a method for detecting bio-inhibitory substances, comprising the steps of: obtaining a test sample; fully mixing the test sample with the aforementioned recombinant bio-luminescent bacterium; and detecting the luminosity of aforementioned recombinant bio-luminescent bacterium and from the decrease of luminosity the level of bio-inhibitory substances in the sample is to be determined.

These and other aspects and advantages will become apparent when the DESCRIPTION below is read in conjunction with the accompanying Examples.

### BRIEF DESCRIPTION OF THE DRAWINGS

FIG. 1 shows the pLWY-sh1A recombinant DNA vector constructed according to the present invention.

FIG. 2 shows the pLWY-fabG recombinant DNA vector constructed according to the present invention.

FIG. 3 shows the use of luminescence phenotype of the recombinant bio-luminescent bacterium for screening work according to the present invention.

FIG. 4 shows the recombinant DNA vector carried by the recombinant bio-luminescent bacterium according to the present invention.

FIG. 5 shows the luminosity of recombinant bio-luminescent bacterium provided herein versus phenol concentration.

FIG. 6 shows the luminosity of recombinant bio-luminescent bacterium provided herein versus acetonitrile concentration.

FIG. 7 shows the luminosity of recombinant bio-luminescent bacterium provided herein versus zinc ion concentration.

### DETAILED DESCRIPTION OF THE INVENTION

The present invention relates to recombinant DNA vector, recombinant bio-luminescent bacterium and method for detecting bio-inhibitory substances using the foregoing bacterium based on the two component system of bacteria that senses the changes of environmental signals. Such system consists of two components, one is a sensor located at the cytoplasmic membrane of the bacterium; when the sensor reacts to the changes of signal molecules in the environment, such as temperature, osmotic pressure or metal ion, autophosphorylation of histidine occurs. This phosphorylation signal is then transferred to response regulator. Phosphorylation of the response regulator modulates its ability to mediate downstream genes. The response of response regulator to an external stimulus (i.e. bio-inhibitory substances in this invention) is reflected in the physiological gene expression of the bacterium to regulate the bacterial physiology and adapt to the environment. The present invention utilizes a pair of two component systems in Serratia marcescens to mediate genes relating to hemolysin and fatty acid metabolism, which are sh1A and fabG gene, respectively. Each promoter of the two genes couples individually with light-emitting gene as reporter to construct a recombinant DNA vector. Said recombinant DNA vector is then transformed to enteric bacillus. Through the response of the bacterium to external stimulus, the concentration of the toxic substance may be inversely deduced. As such, the bacterium may be used as a monitoring tool for toxicants in wastewater.

It must be noted that as used herein and in the appended claims, the singular forms “a”, “an”, and “the” include plural reference unless the context clearly dictates otherwise. Thus, for example, reference to “the recombinant DNA vector” includes a plurality of such recombinant DNA vectors; reference to “a recombinant bio-luminescent bacterium” is a reference to one or more recombinant bio-luminescent bacteria and equivalents thereof known to those skilled in the art, and so forth.

It should be noted that the sh1A gene promoter used herein represents the full length sequence including the sequence of SEQ ID NO: 3, a fragment, derivative, or degenerate sequence thereof.

It should as well be noted that the fabG gene promoter used herein represents the full length sequence including the sequence of SEQ ID NO: 6, a fragment, derivative, or degenerate sequence thereof.

The aforesaid “fragment sequence” used herein refers to a partial sequence of the aforementioned gene.

The aforesaid “derivative” used herein refers to a fragment having at least 70% sequence homology with the gene sequence disclosed above or fragment with 3′ or 5′-end of aforesaid gene sequence modified with other nucleotides and having at least 70% sequence homology with the gene sequence disclosed above.

The aforesaid “degenerate sequence” used herein refers to the foregoing sequences having nucleotides being partially substituted (30% or less) with other nucleotides.

The aforesaid light-emitting gene refers to a gene that encodes light-emitting protein, such as a fluorescent or luminescent protein. The light-emitting gene can be selected from a group consisting of luciferase gene, green fluorescent protein gene and blue fluorescent protein gene, wherein the luciferase gene is more preferable, luxCDABE gene is even more preferable.

The aforesaid recombinant DNA vector may further include a selective marker, said selective marker being preferably antibiotic-resistant gene. The aforesaid antibiotic-resistant gene can be selected from a group consisting of ampicillin-resistant gene, kanamycin-resistant gene, tetracycline-resistant gene and streptomycin-resistant gene, etc.

The aforesaid recombinant bio-luminescent bacterium as described in the present invention for detecting bio-inhibitory substances can be, for example, but not limited to the enteric bacilli, including Escherichia coli and Serratia marcescens, even more preferably Serratia marcescens.

A further objective of the present invention is to provide a method for detecting bio-inhibitory substances, wherein the aforesaid “bio-inhibitory substances” can be, for example, but not limited to heavy metals, organic substances, metal ions, or other toxicants that inhibit the normal physiological functions of organisms.

The following examples are presented in order to more fully illustrate the preferred embodiments of the invention. They should in no way be construed, however, as limiting the broad scope of the invention. While the invention is described and illustrated herein by references to various specific material, procedures and examples, it is understood that the invention is not restricted to the particular material combinations of material, and procedures selected for that purpose. Numerous variations of such details can be implied as will be appreciated by those skilled in the art.

### Example 1

### Construction of Recombinant DNA Vector and Recombinant Bio-Luminescent Bacterium

Required Chemicals and Apparatus

1. Culture Medium: LB Broth

Dissolve 1% (wt/v) Bacto tryptone (Difco, U.S.A.), 1% NaCl, 0.5% Bacto yeast extract (Difco, U.S.A.) or 2.5% LB base powder (GIBCO/BRL, U.S.A.) in sterile water and sterilize with autoclave for 15 minutes under 121° C.

2. Required Antibiotics

| | Stock | Concentration | |
|---|---|---|---|
| Name | concentration (mg/ml) | (μg/ml) | Supplier |
| Ampicillin | 50, in purified water | 100 | Sigma, U.S.A. |
| Kanamycin | 50, in purified water | 50 | Sigma, U.S.A. |
| Streptomycin | 50, in purified water | 50 | Sigma, U.S.A. |
| Tetracycline | 13, in purified water | 13 | Sigma, U.S.A. |

3. Gel Electrophoresis Reagents
(1) Agar (electro endosmosis agarose)
(2) 1×TAE electrophoresis buffer (40 mM Tris-acetate, 1 mM EDTA, pH 8.0): Diluted from 50×TAE (each liter contains 242 g of Tris base, 57.1 ml of glacial acetic acid and 100 ml of 0.5M EDTA-8.0).
(3)10×tracking dye
   0.25% bromophenol blue, 0.25% xylene cyanol, 0.1 mol EDTA, and 50% glycerol.
(4) Ethidium bromide solution.
(5) Standard DNA molecular weight marker.
   Experimental Process
   (1) Searching for Luminescent Genes

Search for the required luminescent DNA fragment luxCDABE and bacteria containing this fragment using bioinformatic approaches, such as NCBI Nucleotide Database. After obtaining the strain, place it in 30° C. or 37° C. LB broth as described above and add proper antibiotics. After shake culture overnight, 1.5 ml bacterial fluid is transferred into micro centrifuge tube and centrifuged at 13,000 rpm for 2 minutes. Remove the supernatant and decant residual fluid with micropipette tip as much as possible. Add 0.6 ml lysis solution to the precipitate and use micropipette to disperse the precipitate to turn it into suspension. Place the tube under 80° C. to break the bacteria. Add 0.2 ml precipitation solution. Cap the tube and shake up and down repeatedly. Place the centrifuge tube in ice bath and then centrifuge at 12,000 rpm for 15 minutes. Transfer the supernatant with care into another centrifuge tube and add to it isopropanol one time the volume of supernatant. Mix well and centrifuge at 12,000 rpm for 10 minutes. Pour out the supernatant. Wash the precipitate with 70% alcohol twice and centrifuge 1 minute after each wash. After drying the precipitate with SpeedVac, dissolve with 30 μL TE-8.0 to obtain the chromosome DNA of the bacterium. Select appropriate restriction enzyme to excise the chromosome DNA to obtain the segment comprising complete luxCDABE genes.

(2) Construction of pLWY-sh1A—Recombinant DNA Vector with sh1A Promoter and luxCDABE Genes

Bacterium with sh1A gene promoter is obtained with bioinformatic approach. After obtaining the chromosome DNA of said bacterium, design appropriate primer (sh1A forward primer, SEQ ID NO: 1 TCTGAATTCGCAGCAGCGCCGGTATAAGCAC; sh1A reverse primer, SEQ ID NO: 2 TACGTAATCGCCAGCGCAGCGGCCAGTT) and carry out polymerase chain reaction (PCR). As shown in Table 1, add successively the following reagents to 0.5 ml micro centrifuge tube.

TABLE 1

| Reactant | Volume (ml) | Final concentration |
|---|---|---|
| DI water | 28.5 | — |
| 10 × PCR buffer | 5 | 1× |
| 10 × dNTP solution | 5 | 200 μM |
| Forward primer | 5 | 0.2 μM |
| Reverse primer | 5 | 0.2 μM |
| Template DNA | 1 | ˜50 pg |
| Taq DNA polymerase | 0.5 | 0.5 U |
| Total volume | 50 | |

Tap the tube wall with finger gently to let the reagents mix well. On PCR reactor, set the reaction time 94° C., 1 min; 60° C., 40 sec; 72° C., 30 sec. Subject the sh1A DNA fragment obtained from PCR (SEQ ID NO: 3 CA GCAGCGCCGG TATAAGCACC GGCGCCACAT TGCGTTATCA GCGAGCGCTG GTCGATCTGG AGGTCAGCCG CGGCGGATT TTGTCTAACC ACGCTACGCC GGAAGATCCC GTTCAGGTGT TGGCCCGCTT TTCTTACACC TTTTAATCAA CAGTTTTGCA GGACCCCACG GCAATATACG GAGAGACATG GATGAAAAAT AATAACTTCA GACTTTCGGC GGCAGGCAAA CTGGCCGCTG CGCTGG) to electrophoresis to purify the desired product.

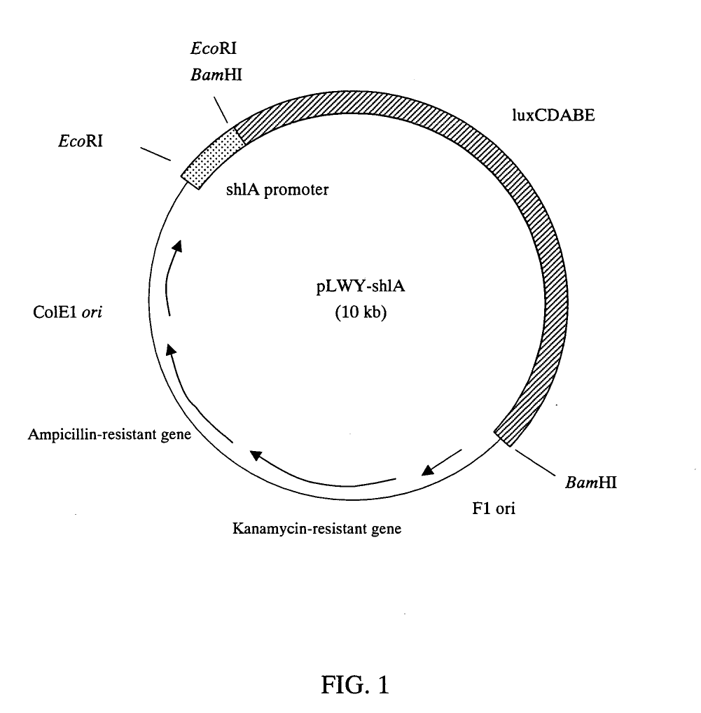

After verifying the luxCDABE genes and sh1A promoter sequence, construct these two DNA fragments onto an appropriate vector and thus the recombinant DNA vector, pLWY-sh1A, is to be obtained as shown in FIG. 1.

(3) Construction of pLWY-fabG—Recombinant DNA Vector with fabG Promoter and luxCDABE Genes

The method used in this step is substantially the same as that in step (2); design appropriate primer (primers for amplifying fabG gene promoter are fabG forward primer—SEQ ID NO: 4 CCGGAATTCGCCGGTTACCAATGCCACTTT; and fabG reverse primer, SEQ ID NO: 5 TAATACGTAGGGGGAGCGAAACATGCAG) and carry out polymerase chain reaction (PCR). Set the reaction time 94° C., 1 min; 60° C., 40 sec; 72° C., 30 sec. Subject the fabG DNA fragment obtained from PCR (SEQ ID ATGEGCAGAG CCATAGCCAC GGCGGCCATC ATGCTTCCGC TTTGCAGCGG GTACATCAGG CGGCGGCGAC GGATGCTTA CGGCTGCGCC AAGTCGCGCA TTAAAAAGCA GCTGCGCCGC CTGGCTCAGC TTTGAAGCGG TTTTTAGGGA GCCGCTCTAT ACTCTGACGG GCATACGGGG CGCCTGCGCG CAGGCCGCTG TGTTGATATC GCRAACCAGA GAAAAAACAG TATGCGAAA GTGGCATTGG TAACCGGCGC AAGC)) to electrophoresis to purify the desired product.

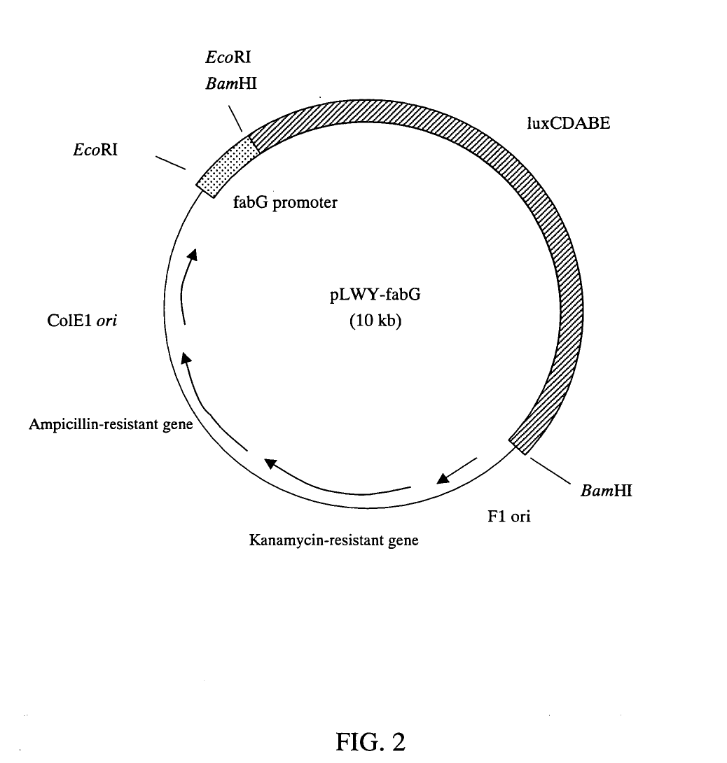

After verifying the luxCDABE genes and fabG promoter sequence, construct these two DNA fragments onto an appropriate vector and thus the recombinant DNA vector, pLWY-fabG, is to be obtained as shown in FIG. 2.

(4) Establishment of Recombinant Bio-Luminescent Bacterium

Transform the recombinant DNA vectors, pLWY-sh1A and pLWY-fabG, obtained in step (2) and step (3) to competent cells. The competent cells should be prepared 16 to 20 hours prior to the transformation procedure by the following steps: inoculate enteric bacilli, such as Escherichia coli or Serratia marcescens on LB agar plate and culture overnight at 37° C. Pick up one colony from LB agar plate to seed it in 1 ml LB medium. Vortex to disperse the bacteria and dilute it with 40 ml LB medium. Shake culture under 37° C. for 2 to 3 hours until cell count reaches 4˜7×10⁷/ml. Harvest the bacteria and place on ice for 10 to 15 minutes. Centrifuge at 750 to 1,000 g under 4° C. for 12-15 minutes. Remove the supernatant and add in ice-cold 0.1 M CaCl₂ of 1/3 the volume of bacterial liquid. Shake to mix well and stood on ice for 10-15 minutes. Repeat the soaking and washing two to three times to obtain complete reaction. Add in ice-cold 0.1 M CaCl₂ of 1/25 the volume of bacterial liquid, mix well, and the competent cells are produced. Next transform pLWY-sh1A and pLWY-fabG respectively to the competent cell by the following steps: place <10 μl DNA in micro centrifuge tube and pre-cool on ice. Take another micro centrifuge tube without adding any DNA (as negative control) and place it on ice for pre-cooling. Add into each micro centrifuge tube 200 μl competent cells. After vortexing and mixing, stand the tube on ice for 20 to 40 minutes and followed by heat shock under 42° C. for 90 sec. After standing on ice for 2 minutes, add in 800 μl LB and shake culture under 37° C. for 30 to 60 minutes. Afterwards, plate appropriate amount of bacterial fluid on LB agar plate containing proper antibiotics, and let the bacterial liquid to be absorbed completely. Put the culture dish upside down and culture under 37° for 16-20 hours. Observe the morphology of colonies on each plate and count the number of colony. Antibiotics is used for a preliminary screening in the hope to obtain colonies that carry the desired recombinant DNA vectors. The choice of antibiotics may vary based on the antibiotics-resistant genes carried by the vector. The commonly used antibiotics include ampicillin, kanamycin, tetracycline and streptomycin.

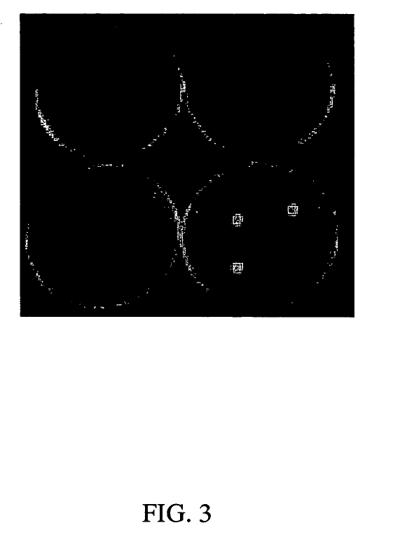

To further confirm that the bacteria obtained are the recombinant bio-luminescent bacteria, the luminescence phenotype is utilized for screening. The results are shown in FIG. 3. The three white dots at the lower right corner of the plate are the colonies of recombinant bio-luminescent bacterium provided herein. Moreover, polymerase chain reaction may be employed to preliminarily screen undesired colonies according to the steps described below: after mixing the reactants as shown in Table 2, set proper reaction time on the PCR reactor. After the reaction is completed, determine the DNA sequencing with capillary electrophoresis.

TABLE 2
Reactants for sequence reading

| Reactant | Volume (ml) |
|---|---|
| DI water | 9 |
| Sequencing premix | 5 |
| Forward primer | 1 |
| Template DNA | 5 |
| Total volume | 20 |

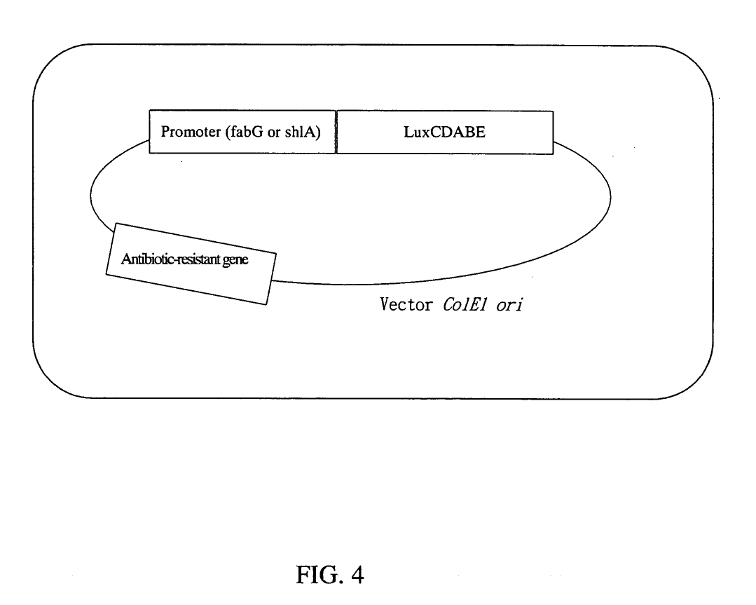

After verifying the desired sequence, the construction of recombinant bio-luminescent bacterium as shown in FIG. 4 is completed. The aforesaid bacteria carrying pLWY-sh1A recombinant DNA vector have been preserved at the Culture Collection and Research Center of the Food Industry Research and Development Institute (No. BCRC940434) since Oct. 17, 2003. The bacteria carrying pLWY-fabG recombinant DNA vector have been preserved at the Culture Collection and Research Center of the Food Industry Research and Development Institute (No. BCRC940433) since Oct. 17, 2003.

### Example 2

### Utilizing Recombinant Bio-Luminescent Bacterium of the Present Invention to Detect Bio-Inhibitory Substance (Phenol) in Wastewater

The technique developed in this invention that allows speedy detection of bio-inhibitory substances in wastewater is based on the principle that such substance poses a negative effect on the luminescent ability of recombinant luminescent bacteria, pLWY-fabG (in Serratia marcescens) and pLWY-sh1A (in Serratia marcescens) as provided herein. The detailed process for detecting bio-inhibitory substances in wastewater using the foregoing recombinant bio-luminescent bacteria are described below: 1. Culture the constructed foregoing recombinant bio-luminescent bacteria for 18-24 hours (O.D. >2); 2. Dilute the bacterial liquid at 1:100 with fresh LB broth the next day; 3. Shake culture at 225 rpm under 30° C. for one hour; 4. Mix 200 μl of bacterial liquid with 20 μl of test sample; mix well and let them react for 15 minutes; and 5. Use luminometer Berthold Lumat LB9507 to detect luminosity for 20 sec and the results are shown in relative light unit (RLU).

To preclude environmental error, each batch of test samples must include purified water to be treated as a positive control with RLU set as 100% for a standard reference. Given that the RLU of negative control (LB broth) is 0, it was not measured. The test results of samples are expressed as a percentage of the positive control. In this test, the amount of bacterial liquid used was 200 μl and that of test sample was 20 μl. Therefore the concentration of test sample was diluted to 1/11 of the original liquid at the time of testing. The concentrations of samples shown in the table are the detected concentration.

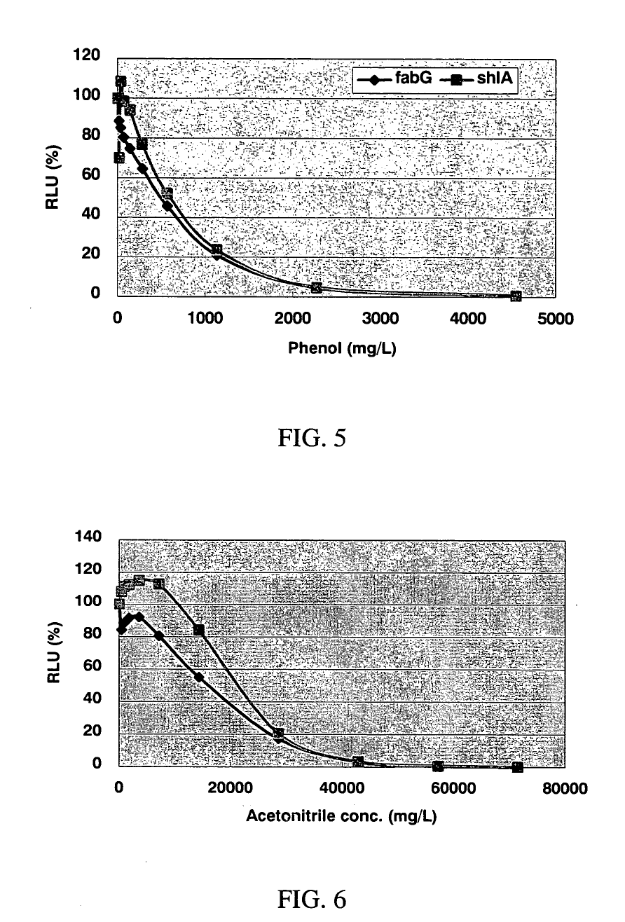

In this example, the recombinant bio-luminescent bacterium provided herein was utilized to detect the concentration of phenol in wastewater. As shown in FIG. 5, the RLU values of the luminescent bacterium indicate the presence of phenol, and the higher is the phenol concentration, the lower is the RLU value. Within a certain concentration range, the concentration of phenol in wastewater and the luminosity of recombinant bio-luminescent bacterium provided herein exhibit a linear relationship, demonstrating the successful application of this invention in detecting the bio-inhibitory substances in wastewater.

### Example 3

### Utilizing Recombinant Bio-Luminescent Bacterium of the Present Invention to Detect Bio-Inhibitory Substance (Acetonitrile) in Wastewater

In this example, the same steps and conditions as in Example 2 were employed to test the level of acetonitrile in wastewater. The results, as shown in FIG. 6 indicate that the RLU values of the luminescent bacterium could indeed reveal the presence of acetonitrile in wastewater, and the higher is the acetonitrile concentration, the lower is the RLU value. Within a certain concentration range, the concentration of acetonitrile in wastewater and the luminosity of recombinant bio-luminescent bacterium provided herein exhibit a linear relationship, demonstrating the successful application of this invention in detecting the bio-inhibitory substances in wastewater.

### Example 4

### Utilizing Recombinant Bio-Luminescent Bacterium of the Present Invention to Detect Bio-Inhibitory Substance (Zinc Ion) in Wastewater

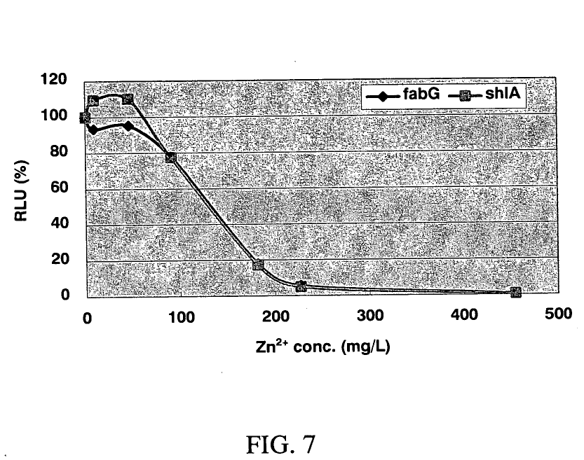

In this example, the same steps and conditions as in Example 2 were employed to test the level of zinc ion in wastewater. The results as shown in FIG. 7 indicate that the RLU values of the luminescent bacterium could indeed reflect the presence of zinc ion in wastewater, and the higher is the zinc ion concentration, the lower is the RLU value. Within a certain concentration range, the concentration of zinc ion in wastewater and the luminosity of recombinant bio-luminescent bacterium provided herein exhibit a linear relationship, demonstrating the successful application of this invention in detecting the bio-inhibitory substances in wastewater.

Based on the results in the three examples above that utilized recombinant bio-luminescent bacterium to detect three bio-inhibitory substances in wastewater, namely phenol, acetonitrile and zinc ion, the RLU values of the luminescent bacterium could indeed reflect the presence of those substances in wastewater, and the higher is the concentration of the bio-inhibitory substance, the lower is the luminosity value. For different bio-inhibitory substances, their concentration within a certain range had a linear relationship with the RLU value of the recombinant bio-luminescent bacterium, demonstrating that this invention could be successfully applied in the detection of bio-inhibitory substances in wastewater.

The preferred embodiment of the present invention as disclosed above is not meant to limit this invention. All modifications and alterations made by those skilled in the art without departing from the spirits of the invention and appended claims shall remain within the protected scope and claims of the invention.

1. A recombinant DNA vector, comprising:
a promoter, comprising full length sh1A gene promoter or fabG gene promoter, a fragment, derivative or degenerate sequence thereof; and
a reporter, comprising a light-emitting protein encoding gene positioned in-frame downstream of said promoter.

2. The recombinant DNA vector according to claim 1, wherein said sh1A gene promoter contains sequence of SEQ ID NO: 3, a fragment, derivative or degenerate sequence thereof.

3. The recombinant DNA vector according to claim 1, wherein said fabG gene promoter contains sequence of SEQ ID NO: 6, a fragment, derivative or degenerate sequence thereof.

4. The recombinant DNA vector according to claim 1, wherein said light-emitting protein encoding gene is selected from a group consisting of luciferase gene, green fluorescent protein gene and blue fluorescent protein gene.

5. The recombinant DNA vector according to claim 4, wherein said light-emitting protein encoding gene is luciferase gene.

6. The recombinant DNA vector according to claim 5, wherein said luciferase gene is luxCDABE gene.

7. The recombinant DNA vector according to claim 1, wherein said vector further includes a selective marker.

8. The recombinant DNA vector according to claim 7, wherein said selective marker is an antibiotic-resistant gene.

9. The recombinant DNA vector according to claim 8, wherein said antibiotic-resistant gene is selected from a group consisting of ampicillin-resistant gene, kanamycin-resistant gene, tetracycline-resistant gene and streptomycin-resistant gene.

10. A recombinant bio-luminescent bacterium for detecting bio-inhibitory substances carrying any of recombinant DNA vector of claims 1, said recombinant bio-luminescent bacterium can sense the concentration of bio-inhibitory substance, wherein the higher is the concentration of bio-inhibitory substance, the lower is the luminosity of said bacterium.

11. The recombinant bio-luminescent bacterium according to claim 10, wherein said bacterium is enteric bacillus.

12. The recombinant bio-luminescent bacterium according to claim 11, wherein said bacterium is Serratia marcescens.

13. A method for detecting bio-inhibitory substances, comprising the steps of:
obtaining a test sample;
fully mixing the test sample with the recombinant bio-luminescent bacterium of claim 10; and
detecting the luminosity of said recombinant bio-luminescent bacterium and from the decrease of luminosity, the level of bio-inhibitory substances in the sample is to be determined.
